(19) United States
(12) Patent Application Publication
(10) Pub. No.: US 2005/0058712 A1
(43) Pub. Date: Mar. 17, 2005

(54) Aqueous dispersions of at least one biodegradable polymer

(76) Inventors: Michel Serpelloni, Beuvry Les Bethune (FR); Leon Mentink, Roubaix (FR); Joel Bernaerts, Labeuvriere (FR)

Correspondence Address: STURM & FIX LLP, 206 SIXTH AVENUE, SUITE 1213, DES MOINES, IA, 50309-4076, US

(21) Appl. No.: 10/936,285

(22) Filed: Sep. 8, 2004

(30) Foreign Application Priority Data
Sep. 12, 2003 (FR) 03 10743

(51) Int. Cl. A61K 9/14

(52) U.S. Cl. 424/486

(57) ABSTRACT

The present invention relates to aqueous dispersions of at least one biodegradable polymer comprising an organic phase dispersed in a dispersing aqueous phase and free of volatile organic compounds, characterized in that the said organic phase comprises at least one viscosity-reducing agent with a solubility parameter of between 15 and 28 (J.cm⁻³)^{0.5}, preferably between 16 and 23 (J.cm⁻³)^{0.5} and even more preferably between 18 and 21 (J.cm⁻³)^{0.5}, and a hydrogen bonding parameter δH of between 3.5 and 15 (J.cm⁻³)^{0.5}, preferably between 4 and 13 (J.cm⁻³)^{0.5} and even more preferably between 5 and 10 (J.cm⁻³)^{0.5}, and at least one biodegradable polymer in a polymer/viscosity-reducing agent weight ratio of from 99.8/0.2 to 60/40, preferably between 97/3 and 70/30 and more preferably between 92/8 and 70/30.

The present invention relates to aqueous dispersions of at least one biodegradable polymer, which comprises an organic phase dispersed in a dispersing aqueous phase and which are free of volatile organic compounds, the dispersed organic phase of which comprises at least one viscosity-reducing agent.

The invention relates more particularly to aqueous dispersions of at least one biodegradable polymer, in which the viscosity-reducing agent is characterized by its solubility parameter, its capacity to form hydrogen bonds and its specific weight ratio with the said biodegradable polymer.

For the purposes of the invention, the terms “aqueous polymer dispersions” or “latex” mean colloidal dispersions of polymers in an aqueous phase, i.e. dispersions of polymer microparticles suspended in an aqueous phase, which are also occasionally known as polymer suspensions or polymer emulsions.

For the purposes of the invention, the term “biodegradable polymer” means a polymer intended to be degraded by the action of microorganisms, especially soil microorganisms, but also by the action of natural agents, especially water.

Also for the purposes of the invention, the term “viscosity-reducing agent” (or “plasticizer”) means a heavy organic solvent, which, when incorporated into polymers, at least partially destroys the interactions between the chains responsible for the high viscosity and mechanical cohesion of the said polymers.

The viscosity-reducing agent is characterized by its solubility parameter and its capacity to form hydrogen bonds.

The solubility parameters (so called “Hildebrand” parameters) reflect, specifically, the force of attraction existing between the molecules of the solvent and of the polymer, and more particularly the variation in density of the cohesion energy of the solvent, i.e. the energy required to vaporize it. The solubility parameter units are thus expressed at 25° C. and in (J.cm⁻³)^{0.5} or in (MPa)^{1/2} (in which 1 (J.cm⁻³)^{0.5}=1 (MPa)^{1/2}) .

Moreover, the viscosity-reducing agent is also characterized by its capacity (weak, moderate or strong) to form hydrogen bonds, according to the Hansen parameter (also expressed at 25° C. and in (J.cm⁻³)^{0.5}).

For the purposes of the invention, the term “volatile organic compound” means any organic compound with a vapour pressure of 13.3 Pa or more at a temperature of 25° C. or with a corresponding volatility under given conditions.

The viscosity-reducing agent selected for the purposes of the invention is therefore not a volatile organic compound.

The absence of volatile organic compound in the aqueous dispersions of the invention is reflected by a volatile organic compound content that does not exceed that of the ingredients of the said aqueous dispersions, i.e. not more than 5000 ppm, preferably not more than 1000 ppm and even more preferably not more than 500 ppm.

The present invention also relates to the process for manufacturing these particular aqueous dispersions.

Aqueous polymer dispersions are used in fields as varied as adhesives, inks, paints, seed or grain coating systems, paper coating, films, carpet backings or mattress foams.

However, they are also of interest in the pharmaceutical industries (for drug delivery, medical testing kits and surgical gloves) or in cosmetics.

The conventional manufacture of aqueous polymer dispersions, in replacement for natural latex, is based mainly on processes of direct emulsion polymerization of synthetic monomers of styrene, ethylene, propylene, vinyl alcohol or acrylamide type.

These synthetic polymer emulsions are used, for example, by the casting technique, for coating supports of cardboard or paper type in order to give them water-resistant properties.

Their application as a thin coat onto the inner face of paper packagings, especially for milk or fruit juices, has led, however, to the production of packagings that are difficult to recycle and to biodegrade during ground burial treatments.

In order to overcome this problem, aqueous polymer dispersions prepared from biodegradable polymers have thus become the subject of intensive research and development.

Initial techniques consisted in using biodegradable polymers in unmodified form, for example in melt form, which necessitates working them at high temperature, with the drawback of preparing films that are excessively thick, or in using them in extrusion/drawing techniques to generate thinner films, but these techniques are expensive and material- and energy-intensive.

In patent application JP 4 334 448, for the manufacture of water-resistant films, the approach adopted is rather to spray the surface of the paper with a lactic acid polymer dissolved in a volatile organic solvent or to immerse the said paper in an organic solvent containing polylactic acid.

Although the choice of a lactic acid polymer instead of a synthetic resin contributes towards resolving the problem of biodegradability of the polymers and consequently of the packagings, it is not entirely satisfactory.

Specifically, the use of volatile organic compounds as solvents to deposit the polylactic acid or its derivatives on the surface of a substrate entails great complexity of handling as a result of the mandatory precautions imposed by the toxicity of the volatile organic solvent (flammable solvents, risks of explosion, inhalation by the operatives, environmental pollution, etc.).

Furthermore, it is known that this solution is not technologically satisfactory, since the technique of film formation by surface deposition of a solution of biodegradable polymers prepared from lactic acid polymers is again penalized by the difficulties encountered in obtaining films that are sufficiently water-resistant compared with those prepared from synthetic polymers.

In an attempt to solve these water-resistance difficulties, patent application JP 10-101 911 describes the production of a resistant biodegradable film, this time starting with an emulsion comprising a dispersion of fine emulsified globules of polylactic acid containing an anionic emulsifier.

However, this technical solution has the drawback of still requiring the use of a volatile organic solvent such as methylene chloride, chloroform, dioxane or acetonitrile to disperse the lactic acid polymer as an emulsion.

Patent application JP 2001-11294 proposes biodegradable aqueous dispersions comprising, along with a biodegradable resin of aliphatic polyester type, a mixture of a cationic macromolecular compound with a molecular weight (MW) of at least 300 000 or an anionic macromolecular compound with an MW of at least 1 000 000 and polyvinyl alcohol (PVA).

The drawback in this case is the use of a non-biodegradable mixture of cationic or anionic macromolecular compounds/PVA. Moreover, it is also proposed to add other agents, such as thickeners, flow index modifiers, etc.

It is also still obligatory to use as solvent a large excess of volatile organic solvent, which necessitates the use of a subsequent step which is stripping off under high vacuum a large proportion of this organic solvent. It is moreover known that it is not possible to obtain a solvent-free emulsion, save at the risk of breaking the emulsion.

This biodegradable emulsion is barely satisfactory, given the number of manipulations to be performed and the fact that a very precise equilibrium of the components needs to be adhered to in order to obtain an industrially advantageous product.

Finally, these biodegradable aqueous dispersions are of only limited stability, i.e. they are stable for not more than 1 to 2 months at 20° C. or at 40° C., and, as a result, they cannot be used for many applications in which the required stability times are longer.

Patent application JP 2001-303 478 describes a process for manufacturing sheets of paper coated with a layer of biodegradable plastic. It is desired to give the paper mechanical strength, printability and water-resistance properties, and also stability over time. The recommended solution consists in applying a biodegradable plastic emulsion onto a support sheet of paper in order to deposit thereon a layer of biodegradable plastic.

The biodegradable plastic emulsions are prepared by dissolving a lactic acid polymer in a volatile organic compound, i.e. ethyl acetate, and then adding an emulsifier, such as fatty acid salts, carboxylate ethers, alkenyl succinates, alkyl sulphates, etc.

This solution is labour-intensive and complex, and requires the addition of many ingredients, both into the organic phase and into the dispersing aqueous phase, and still in the presence of volatile organic solvents.

In conclusion, all the patent applications mentioned above have three major drawbacks:
it is indispensable to use volatile organic solvents to dissolve the biodegradable polymers, irrespective of the preparation process used, which therefore does not make it possible to avoid the problems of toxicity, cost premiums, complexity of the steps used, the obligation to install leak-prevention devices, antideflagration devices or devices for recovering the volatile organic solvents, and their inevitable environmental impact,
the use of volatile organic solvents can only lead to aqueous dispersions with a low dry matter (DM) content,
it is necessary to add various stabilizers, emulsifiers, thickeners, etc., the synthetic nature of which is detrimental to the biodegradability of the aqueous dispersion obtained.

To illustrate the attempts made in the prior art to overcome the problems of using volatile organic solvents, patent application EP 1 302 502 proposes a process for manufacturing a biodegradable polyester dispersion with a high dry matter (DM) content and a high viscosity. The process comprises treating at high temperature and high pressure, by extrusion, a blend of biodegradable polyesters in the melt with an aqueous emulsifier having a certain surface tension and with certain additives, so as to obtain an aqueous dispersion with a DM of at least 40% and a viscosity of at least 1000 mPa.s and preferably between 1500 and 10 000 mPa.s. The process comprises melting the biodegradable polyesters in the extruder and then introducing therein the aqueous emulsifier downstream of the melt. The mixture is then blended at a temperature of less than or equal to 100° C.

If the biodegradable polyesters have a melting point of greater than or equal to 100° C., it is proposed to increase the working pressure of the extruder or to add plasticizers to lower the said melting point. However, neither the type, the nature (in terms of solubility parameter values and capacity to form hydrogen bonds), nor the amount of plasticizers to be used is specified. No proposals are given for the said plasticizers.

This process also has two major drawbacks:
the first drawback lies in the fact that the process is aimed only at obtaining very viscous aqueous dispersions, which is the only way, according to the teaching of the said patent application EP 1 302 502, to obtain an emulsion that is stable over time.

It is, specifically, a matter of preventing the largest particles from sedimenting out and consequently leading to a lowering of the DM of the emulsion by phase separation.

This solution is not satisfactory since it leads to viscous emulsions that are difficult to handle and makes it difficult to obtain thin films. Specifically, excessively viscous solutions lead to thick films, of heterogeneous thickness, which crack and consume a lot of material.

A viscous aqueous dispersion also does not promote the coalescence of the polymer particles in suspension during the preparation of films (problem of steric bulk) and reduces the mechanical strength of the films.
The second drawback is associated with the nature of the melt/blending process of the biodegradable polyester itself in an extruder.

Specifically, the extrusion operation leads to intense shear of the blended melt, which alters the structure and the intrinsic properties of the polymer. The films made with this aqueous dispersion are mechanically weaker and less rigid. This shear also leads to an increase in hydrophilic end groups and an increase in the mobility of the polymer chains. This increase in hydrophilicity is then reflected by an increase in the diffusion of water into the polymer, which increases the rates of hydrolysis and degradation.

A quite similar process for preparing aqueous dispersions by extrusion, having these same drawbacks, is described in U.S. Pat. No. 4,502,888.

The process described in the said patent applies only to cellulose-based biodegradable polymers combined with long-chain fatty acids, and only allows the production of aqueous dispersions with a low dry matter content (of about 30%).

Patent application WO 97/49762 describes an aqueous dispersion of biodegradable polymers that contains esterification-modified starches, dispersed in an aqueous phase, and a solvent-free process for manufacturing the said aqueous dispersions.

The process includes the step of preparing a molten blend, at a temperature of from 100 to 180° C., of polymers and plasticizers, and then in keeping this blend stirred at high temperature, followed by gradually adding water and dispersants.

It is noted, however, that although aqueous dispersions of biodegradable polymers with a high dry matter content are obtained without volatile organic solvents, the said patent application teaches that this can only be achieved:
by adding plasticizer in very large amount, or even in excess relative to the biodegradable polymers (in point of fact, between 75 parts and 175 parts of plasticizer need to be introduced per 100 parts of biodegradable polymers), which necessarily leads to alteration of the properties imparted by the said polymers,
by maintaining, just as in patent application EP 1 302 502, a high viscosity of the said aqueous dispersions (greater than 1200 mPa.s),
which leads to the production of aqueous dispersions containing very small amounts of polymers, given the need to introduce the plasticizer in excess.

Moreover, it is stated that the melt phase may be prepared in an extruder, which leads to the same problems of alteration of the polymers used as those mentioned previously for the discussion of the teaching of EP 1 302 502.

From the foregoing text as a whole, it is seen that there is an unsatisfied need for an aqueous dispersion of a biodegradable polymer, which is stable over time, free of volatile organic-compounds, of high dry matter content and low viscosity, comprising at least one biodegradable polymer with conserved intrinsic properties, and which may comprise only biodegradable ingredients.

The Applicant Company has, to its credit, reconciled all these objectives, which were hitherto considered to be difficult to reconcile, by conceiving and developing, after extensive research, novel dispersions of biodegradable polymers.

The aqueous dispersions of at least one biodegradable polymer comprising an organic phase dispersed in a dispersing aqueous phase and free of volatile organic compounds, in accordance with the invention, are characterized in that the said organic phase comprises:
at least one viscosity-reducing agent with a solubility parameter of between 15 and 28 (J.cm⁻³)^{0.5}, preferably between 16 and 23 (J.cm⁻³)^{0.5} and even more preferably between 18 and 21 (J.cm⁻³)^{0.5}, and a hydrogen bonding parameter δH of between 3.5 and 15 (J.cm⁻³)^{0.5}, preferably between 4 and 13 (J.cm⁻³)^{0.5} and even more preferably between 5 and 10 (J.cm⁻³)^{0.5},
at least one biodegradable polymer in a polymer/viscosity-reducing agent weight ratio of from 99.8/0.2 to 60/40, preferably between 97/3 and 70/30 and more preferably between 92/8 and 70/30.

The aqueous dispersions of at least one biodegradable polymer in accordance with the invention have a volatile organic compound content that does not exceed that of the ingredients of the said aqueous dispersions, i.e. not more than 5000 ppm, preferably not more than 1000 ppm and even more preferably not more than 500 ppm.

These aqueous dispersions thus have none of the drawbacks of the preparations of the prior art prepared with volatile organic compounds.

The aqueous dispersions of at least one biodegradable polymer in accordance with the invention then have in their dispersed organic phase a viscosity-reducing agent with a particular viscosity parameter and a particular hydrogen bonding parameter.

The Applicant Company has found, after numerous tests, that it is wise to select, for the viscosity-reducing agents, this particular range of solubility and hydrogen bonding parameters so as to make them highly compatible with the very large majority of biodegradable polymers with which they will be combined, thus making it possible to obtain:
a better viscosity-reducing effect on the bio-degradable polymers,
very high homogeneity of the organic phase, even at very high temperature (absence of phase separation),
an improvement in the coalescence of the particles of the aqueous dispersion, which facilitates their use at relatively low temperatures,
an improvement in the film-forming properties of the biodegradable polymers, and
an improvement or an adjustment after coalescence of the elongation at break of the films, their flexibility and their elasticity.

These parameters also make it possible to select viscosity-reducing agents capable of controlling the aptitude of certain biodegradable polymers to crystallize.

The Hildebrand parameters (solubility) and Hansen parameters (hydrogen bonding capacity) of the viscosity-reducing agents are conventionally given in tables available to those skilled in the art.

Reference may be made in particular to the article by H. Burrell entitled Solubility parameters for film formers in Official Digest, October 1955, pp. 726-758 and the article by A. Barton entitled Solubility parameters in Chemical Reviews, 1975, vol. 75, No. 6, pp. 731-753. More complete tables may be found in the publication entitled CRC Handbook of solubility parameters and other cohesion parameters by Allan Barton, CRC Press, Inc. Boca Raton, Fla., 2nd edition, 1991, pp. 94-110.

For the solubility parameter, it is also known that it can be determined by simple calculation from direct measurements, in particular of the latent heat of vaporization of the compound of interest, of its boiling point or from empirical equations such as the Hildebrand equation.

The viscosity-reducing agents of the aqueous dispersions in accordance with the invention should thus have a solubility parameter of between 15 and 28 (J.cm⁻³)^{0.5}, preferably between 16 and 23 (J.cm⁻³)^{0.5} and even more preferably between 18 and 21 (J.cm⁻³)^{0.5}, and a hydrogen bonding parameter δH of between 3.5 and 15 (J.cm⁻³)^{0.5}, preferably between 4 and 13 (J.cm⁻³)^{0.5} and even more preferably between 5 and 10 (J.cm⁻³)^{0.5}.

The viscosity-reducing agents in accordance with the invention are thus chosen from the range of heavy organic solvents with a “moderate” capacity to form hydrogen bonds with the biodegradable polymer.

It has been shown by the Applicant Company that viscosity-reducing agents with solubility and/or hydrogen bonding parameters outside these values, for instance glycerol, which has a solubility parameter of 33.8 (J.cm⁻³)^{0.5} and a hydrogen bonding parameter of 29.3 (J.cm⁻³)^{0.5} (high capacity to form hydrogen bonds), which is, in fact, recommended for plasticizing biodegradable polymers, do not make it possible to significantly reduce the viscosity at elevated temperature of the polymers, occasionally even having a tendency to increase it, or separate out during the cooling of these polymers after high-temperature blending.

This is likewise the case for products that are too apolar, such as long-chain free fatty acids, which do not have solubility parameters in the ranges in accordance with those required for the invention.

It has thus been demonstrated by the Applicant Company that long-chain free fatty acids (containing at least 14 carbon atoms), such as oleic acid, linoleic acid, palmitic acid, stearic acid, etc., are generally immiscible with the biodegradable polymers, even at elevated temperature, and do not constitute viscosity-reducing agents that are sufficiently efficient within the context of the invention.

The aqueous dispersions of at least one biodegradable polymer in accordance with the invention then have in their organic phase at least one biodegradable polymer in a polymer/viscosity-reducing agent weight ratio of from 99.8/0.2 to 60/40, preferably between 97/3 and 70/30 and more preferably between 92/8 and 70/30.

It has been found by the Applicant Company that these polymer/viscosity-reducing agent weight ratios are advantageously chosen so as to allow:
better softening of the films,
better resistance to mechanical impacts,
better conservation of the cohesive properties of the film obtained, and
an increase in the water resistance.

The Applicant Company has thus shown that the use of viscosity-reducing agents in these proportions allows a more effective plasticizing effect.

The Applicant Company has moreover found that the absence of viscosity-reducing agents makes it difficult to prepare aqueous dispersions or to obtain aqueous dispersions that are stable over time. On the other hand, the excessive presence of viscosity-reducing agents, i.e. more than 40%, greatly alters the film-forming properties of the biodegradable polymers.

The aqueous dispersions according to the invention advantageously have a viscosity at 20° C. of between 50 and 5000 mPa.s, preferably between 50 and 2000 mPa.s, even more preferably greater than or equal to 100 mPa.s and strictly less than 1000 mPa.s.

The viscosity of the aqueous dispersions in accordance with the invention is measured by Brookfield viscometry or equivalent, according to the conditions specified by the manufacturer.

The Applicant Company has, to its credit, proposed aqueous dispersions of at least one biodegradable polymer that has high stability while at the same time having a low viscosity.

This results, as will be established below, from the choice of the particular nature and the suitable amount of the viscosity-reducing agent contained in their organic phase.

For example, in patent application WO 97/49762, when the biodegradable polymers are starch esters, use is made of a biodegradable polymer/plasticizer weight ratio of 57/43, and in the majority of cases of 36/64.

Surprisingly and unexpectedly, the aqueous dispersions of at least one biodegradable polymer according to the invention do not, on the contrary, contain more than 40% of viscosity-reducing agent in their organic phase, and yet have a low viscosity that generally does not exceed 5000 mPa.s.

Moreover, to the Applicant Company's knowledge, no stable aqueous dispersions of biodegradable polymers free of volatile organic compounds and having a viscosity strictly less than 1000 mPa.s exist in the prior art.

The aqueous dispersions in accordance with the invention advantageously have a dry matter content of between 25% and 70% by weight, preferably between 35% and 65% by weight and even more preferably between 40% and 60% by weight.

In contrast with what is deplored in the prior art, it is thus possible to propose, by virtue of the present invention, aqueous dispersions of fluid biodegradable polymers (with a viscosity generally less than 5000 mPa.s and better still strictly less than 1000 mPa.s) that moreover have a high dry matter content.

The aqueous dispersions in accordance with the invention advantageously have a total content of biodegradable polymers and of viscosity-reducing agent of at least 80% by weight of the dry matter of the said aqueous dispersions, preferably at least 90% by weight and even more preferably at least 95% by weight.

The total content of biodegradable polymers in these aqueous dispersions is advantageously at least 50% by weight of the DM of the said aqueous dispersions, preferably at least 65% by weight, even more preferably at least 70% by weight and better still at least 75% by weight.

The biodegradable polymers are compounds that are virtually insoluble in water but are often also relatively insoluble in volatile organic solvents. It is for this reason that the conventional techniques for preparing emulsions of biodegradable polymers using volatile organic solvents cannot contain more than 20% of biodegradable polymers, provided that the solvent is not stripped off.

The aqueous dispersions of at least one biodegradable polymer in accordance with the invention are also characterized by their particle size distribution.

The size of these particles is determined on a Beckman Coulter LS 13320 laser granulometer with the liquid module.

The aqueous dispersions in accordance with the invention advantageously have a mean particle size of the particles of the said dispersions of between 0.05 and 10 μm and preferably between 0.5 and 5 μm.

The particle size is entirely advantageous both for obtaining stable aqueous dispersions and so that the films prepared with these aqueous dispersions have entirely satisfactory printability and mechanical strength.

The aqueous dispersions according to the invention also advantageously have a 90% content of particles less than 15 μm, preferably less than 10 μm and even more preferably less than 8 μm in size.

The Applicant Company has moreover found that multimodal compositions of fine particle size distribution allow better filling of the spaces between particles. The resulting emulsions thus have an even lower viscosity (strictly less than 1000 mPa.s) or may have a higher dry matter content.

The Applicant Company has found that the choice of the biodegradable polymer is not a limiting factor, which in itself is remarkable. Specifically, by adhering to the profile of the viscosity-reducing agent in its solubility parameter values, its hydrogen bonding capacity and its weight ratio with the polymer under consideration, any type of biodegradable polymer may be incorporated.

However, the Applicant Company recommends selecting it from the group consisting of biodegradable polymers of polylactate (or PLA), polymalate (or PMA), polyhydroxyalkanoate (or PHA), polycaprolactone (or PCL), polyesteramide (PEA) types, aliphatic copolyesters (PBSA) and aliphatic copolyester-co-terephthalates (PBAT), highly acetylated starches or starches made hydrophobic by introducing fixed fatty chains, taken alone or in combination, in the form of homopolymers or heteropolymers, whether they are linear, branched, crosslinked, dendritic or grafted.

Advantageously, the biodegradable polymers are heteropolymers, preferably di-, tri- or tetrapolymers whose monomers are diols, caprolactones or acids and hydroxy acids chosen from the group consisting of D-lactic acid, L-lactic acid, glycolic acid, tetramethylglycolic acid, malic acid, β-propiolactic acid, butyric acid, valeric acid, phthalic acid, terephthalic acid, succinic acid, adipic acid, sebacic acid, hexanoic acid, octanoic acid, decanoic acid, dodecanoic acid, tetradecanoic acid, hexadecanoic acid and octadecanoic acid.

For the heteropolymers, the Applicant Company recommends selecting at least two monomers to be polymerized such that the first is solid at room temperature and the second is more fluid at this temperature. Aqueous dispersions that are highly suitable for forming films are obtained in this case.

The biodegradable polymers may be random, alternating, sequenced or block heteropolymers.

The Applicant Company has found that the aptitude of the polymers to crystallize strongly was not recommended for the aqueous dispersions in accordance with the invention.

It is preferred to select a biodegradable polymer that is in the said dispersions in amorphous or semi-crystalline form, preferably in amorphous form.

For the purposes of the invention, the “amorphous” form of the biodegradable polymer contains not more than 25% of polymers in crystalline form. The “semi-crystalline” form contains not more than 60% of polymers in crystalline form.

In this respect, a biodegradable polymer of PLA type may be chosen, with a D-lactic content after hydrolysis (or as copolymers) of greater than 10% and preferably between 12% and 88%; of PHA type, with a content of comonomers, for example of hydroxyvalerate, hydroxyhexanoate or hydroxyoctanoate type, of greater than 5% and preferably greater than 10%.

A biodegradable polymer functionalized, preferably with a group of aldehyde, fluoro, carboxylic acid, amine or alcohol type, is optionally chosen.

Finally, the Applicant Company has found that the biodegradable polymers of PHA type sold by the company Procter & Gamble under the brand name Nodax® and the biodegradable polymers of PLA type sold by the company Galactic under the brand name Galastic® L68 are particularly suitable.

The analytical characteristics of the constituent biodegradable polymers of the organic phase of the aqueous dispersions in accordance with the invention are preferably as follows:
an MW of between 10 000 and 1 000 000 d, preferably between 15 000 and 500 000 d and more preferably between 15 000 and 200 000 d,
a glass transition temperature (Tg) of between −70 and +70° C., preferably between −45 and +60° C. and even more preferably between −30 and +20° C.,
a “complex” viscosity at 160° C. of between 5 and 20 000 Pa.s, preferably between 10 and 5 000 Pa.s and even more preferably between 20 and 500 Pa.s.

The glass transition temperature is conventionally determined by differential thermal analysis on a Mettler DSC machine of DSC 821 type.

The “complex” viscosity is a viscosity measured in dynamic mode (under sinusoidal stress) on a TA Instruments rheometer of AR 2000 type, according to the manufacturer's specifications.

As regards the nature of the viscosity-reducing agent, the Applicant Company recommends selecting it from the group of optionally ethoxylated esters of acids and of alcohols, preferably mono-, di- or triesters of organic acids, of carbonic acid and of phosphoric acid, of sugars or of polyols.

It may also be chosen from the group of optionally ethoxylated ethers of sugars, of polyols (especially of isosorbide), of glycol or of phenol, of glycol ether-esters or of epoxidized triglyceride oils.

It is desirable for the agent chosen to be biodegradable and of food grade.

The viscosity-reducing agent also advantageously has a boiling point of greater than 130° C., preferably greater than 150° C. and even more preferably greater than 200° C., which places it in the category of heavy solvents that are particularly suited to their function.

The Applicant Company recommends selecting the viscosity-reducing agent from the group consisting of glycerol triacetate, dimethyl isosorbide, isosorbide, isoidide or isomannide diacetates, dibutyrates, diisobutyrates, dihexylates, diethylhexylates, dioctanoates, didecanoates or didodecanoates, ethyl lactate, butyl lactate, methyl laurate, dibutyl maleate, tributyl citrate, triethyl citrate, bis(2-ethylhexyl) adipate, diisobutyl adipate, dibutyl phthalate, propionic acid, glycerol tributyrate, glycerol triisobutyrate, ethylene glycol dibenzoate, diethylene glycol dibenzoate, propylene glycol dibenzoate, dipropylene glycol dibenzoate, triethylene glycol benzoate, dibutyl sebacate, diisobutyl sebacate, propylene carbonate, polyethylene glycol 400, polyethylene glycol 600, caprolactone diols with a molecular weight in the region of 500, fatty esters of adipic, succinic and glucaric acids such as the products sold by the company Dupont under the brand names DBE® and DBE®-IB, and methyl, ethyl, butyl, hexyl and ethylhexyl esters of vegetable oil.

All these viscosity-reducing agents have a Hildebrand parameter of between 15 and 28 (J.cm⁻³)^{0.5}, preferably between 16 and 23 (J.cm⁻³)^{0.5} and even more preferably between 18 and 21 (J.cm⁻³)^{0.5}, and a hydrogen bonding parameter δH of between 3.5 and 15 (J.cm⁻³)^{0.5}, preferably between 4 and 13 (J.cm⁻³)^{0.5} and even more preferably between 5 and 10 (J.cm⁻³)^{0.5}.

The aqueous dispersions in accordance with the invention are thus characterized in that the blend of biodegradable polymer with the viscosity-reducing agent of the organic phase has a complex viscosity at 160° C. of between 0.05 and 30 Pa.s, preferably between 0.5 and 15 Pa.s and more preferably between 1 and 10 Pa.s.

The aqueous dispersions in accordance with the invention also comprise in their dispersing aqueous phase a stabilizer chosen from the group of polyols, oligosaccharides and polysaccharides derived from celluloses, starches and natural and fermenting gums, in native form or modified, preferably by hydroxy-propylation, hydroxy-ethylation, alkylation or alkyl succinylation, peptides and polypeptides, and optionally modified polyvinyl alcohols.

The choice of a polyol as stabilizer makes it possible, for example, to improve the emulsification of the biodegradable polymer, to increase the boiling point of the aqueous phase above 100° C., to improve the stability of the emulsion, to control the rate of coalescence of the particles and to adjust the hydrophilicity of the film.

The Applicant Company recommends introducing the stabilizer present in the dispersing aqueous phase in a proportion of not more than 20% by weight relative to the total weight of the said aqueous dispersions, preferably not more than 10% by weight and even more preferably not more than 5% by weight.

The preparation of the aqueous dispersions of at least one biodegradable polymer in accordance with the invention, includes the following sequence of steps:
1) preparing the organic phase to be dispersed by melting the blend of biodegradable polymer and of viscosity-reducing agent, at a temperature above the glass transition temperature of the biodegradable polymer so as to obtain a homogeneous melt, the ratio between the biodegradable polymer and the viscosity-reducing agent being between 99.8/0.2 and 60/40, preferably between 97/3 and 70/30 and more preferably between 92/8 and 70/30,
2) totally dissolving at least one stabilizer in the dispersing aqueous phase,
3) simultaneously and continuously introducing the dispersing aqueous phase and the organic phase to be dispersed thus obtained into the blender of an emulsifying unit, the rates of introduction of the two phases having been adjusted beforehand such that the dispersed organic phase represents at least 80% by weight of the dry matter of the said aqueous dispersions, preferably at least 90% by weight and even more preferably at least 95% by weight, and
4) recovering the aqueous dispersions thus obtained.

For the preparation of the aqueous dispersions according to the invention, an emulsifying unit is used, for example an emulsifying unit such as one of those sold by the company Emulbitume (Trégueux-France) comprising:
a turbo-blender,
two assemblies for preparing the organic phase to be dispersed and the dispersing aqueous phase, each comprising a thermally insulated tank, a volumetric pump and a set of thermally insulated pipes.

This type of apparatus allows the preparation of aqueous dispersions at atmospheric pressure and at a flow rate of at least 100 l/h, by simultaneously placing in contact in the blender the organic phase to be dispersed in melt form, comprising, on the one hand, the biodegradable polymer(s) with the viscosity-reducing agent, and, on the other hand, the dispersing aqueous phase comprising the stabilizers.

The first step of the process in accordance with the invention thus is the preparation of the organic phase to be dispersed by melting the blend of biodegradable polymer and viscosity-reducing agent, at a temperature above the glass transition temperature of the biodegradable polymer, so as to obtain a homogeneous melt, the ratio between the biodegradable polymer and the viscosity-reducing agent being between 99.8/0.2 and 60/40, preferably between 97/3 and 70/30 and more preferably between 92/8 and 70/30.

For this first step, no volatile organic compound is thus necessary to dissolve the biodegradable polymer.

A biodegradable polymer stabilized with antioxidants, heat stabilizers, light stabilizers or aqueous-hydrolysis stabilizers is advantageously chosen.

Agents capable of blocking any free carboxylic functions of the biodegradable polymer, for instance polycarbodiimides, may especially be used in this respect.

This involves placing the biodegradable polymer(s) and the viscosity-reducing agent in the said proportions at a temperature that is sufficient to obtain a homogeneous melt.

This temperature is generally 100° C. higher than the glass transition temperature of the biodegradable polymer(s) when they are in amorphous form, and generally 10° C. above the glass transition temperature of the biodegradable polymer(s) when they are in semi-crystalline form.

The homogeneous melt is then placed in the thermally insulated tank provided for this purpose in the emulsifier, at a temperature that is constant and sufficient to allow this melt to be pumped by the machine, as will be illustrated later.

The second step of the process in accordance with the invention includes totally dissolving at least one stabilizer in the dispersing aqueous phase.

The stabilizer is dissolved in water, preferably deionized water. The blend is then stirred until a homogeneous solution is obtained, which is then introduced into the thermally insulated tank provided for this purpose in the emulsifier, where it is maintained at this temperature.

The third step of the process in accordance with the invention includes simultaneously and continuously introducing the dispersing aqueous phase and the organic phase to be dispersed thus obtained into the blender of an emulsifier unit, the rates of introduction of the two phases having been adjusted beforehand such that the dispersed organic phase represents at least 80% by weight of the dry matter of the said aqueous dispersions, preferably at least 90% by weight and even more preferably at least 95% by weight.

The flow rates of the organic and aqueous phases are adjusted as a function of their respective viscosity, so as to obtain the desired organic phase/aqueous phase ratio.

Advantageously, in-line cooling of the emulsified blend thus obtained to room temperature may be performed during this step of the process in accordance with the invention.

Finally, the fourth step includes recovering the aqueous dispersions thus obtained.

The aqueous dispersions of at least one biodegradable polymer in accordance with the invention may be used for their binding/tacky, printability, degradability, water resistance/behaviour, gloss, oil resistance, stain resistance and mechanical strength properties, which makes them destined especially for the fields of textiles, coating fluids, paints, adhesives, renderings and mortar fixatives and coatings/encapsulation.

Other characteristics and advantages of the invention will emerge on reading the non-limiting examples described below.

### EXAMPLE 1

A biodegradable polymer Galastic® L68 in the form of polylactate “pellets”, manufactured and sold by the company Galactic, with a number-average molecular weight of 68 000 d approximately, a polydispersity index of 2.78, a glass transition temperature of 54.9° C., a complex viscosity of 220 Pa.s at 130° C. and of 30 Pa.s at 160° C., a degree of crystallinity close to 0% and a content of D-lactic acid after hydrolysis of 12.4%, is chosen for the organic phase.

The viscosity-reducing agent selected is glycerol triacetate (triacetin) as sold by the company Sigma Aldrich.

The Hildebrand parameter, calculated from the latent heat of vaporization of triacetin (85.74 kJ/mol) or from its boiling point (259° C.), is 21 (J.cm⁻³)^{0.5}.

The Hansen parameter, given in the tables of the CRC Handbook, is 11.2 (J.cm⁻³)^{0.5}.

For the aqueous phase, the stabilizer is the polyvinyl alcohol Poval JP 18Y, with a degree of hydrolysis of 88±1%, a viscosity of 25±2 mPa.s and a purity of greater than 94%, sold by the company TVP Japan Vam & Poval Co. Ltd.

The process for manufacturing the corresponding aqueous dispersion is performed as follows:

1) Preparation of the Organic Phase to be Dispersed:

2400 g of Galastic L68 PLA pellets and 600 g of triacetin (i.e. a ratio between the biodegradable polymer and the viscosity-reducing agent of 80/20) are introduced into an electrically heated tank maintained at 160° C.

When these products have completely melted, the blend is made homogeneous by stirring.

2) Preparation of the Dispersing Aqueous Phase:

240 g of Poval JP 18Y polyvinyl alcohol and 0.5 g of Foamaster PD1 antifoam sold by the company Cognis are dispersed in 4500 g of deionized water at 80° C., with mechanical stirring.

3) Preparation of the Aqueous Dispersion in Accordance with the Invention:

The simultaneous blending of the organic phase to be dispersed and of the dispersing aqueous phase is performed in a laboratory emulsifying unit sold by the company Emulbitume.

The organic and aqueous phases are transferred into their respective thermally insulated tanks thermostatically regulated at 160° C. and at 80° C.

The volumetric pumps are switched on in a closed circuit to adjust the respective temperatures and to control the flow rates so as to obtain a content of dispersed organic phase of 95.4% by dry weight.

After opening the valves, the organic and aqueous phases are simultaneously and continuously conveyed into an Atomix C turbo-blender at 8900 rpm.

An aqueous dispersion having the characteristics given in Table I below is obtained at the outlet of the Atomix C turbo-blender.

TABLE I
Initial characteristics

| | |
|---|---|
| Dry matter (%) | 53.7 |
| pH value | 2.2 |
| Mean diameter (μm) | 3.7 |
| d90 (μm) | 9.3 |
| Brookfield viscosity (mPa · s) | 250 |
| Appearance | milky emulsion |

Characteristics after storage for
2 months at room temperature

| | |
|---|---|
| pH value | 2.2 |
| Brookfield viscosity | 190 |
| Appearance | milky emulsion |

The aqueous dispersion thus obtained according to the invention has a very low viscosity for a high dry matter content, while moreover being very stable over time.

### EXAMPLE 2

Three aqueous dispersions are prepared according to the same protocol as that described in Example 1, starting with:
Galastic® L68 biodegradable polymer,
viscosity-reducing agents such as triacetin, but also diisobutyl adipate, sold by the company Sigma Aldrich (its Hildebrand and Hansen parameters are, respectively, 18.5 (J.cm⁻³)^{0.5} and 7.3 (J.cm⁻³)^{0.5}) and the glycol benzoate mixture sold by the company Velsicol Chemical Limited under the brand name Benzoflex® 2088 (its Hildebrand and Hansen parameters are, respectively, 19.8 (J.cm⁻³)^{0.5} and 9.6 (J.cm⁻³)^{0.5}),
stabilizers such as the polyvinyl alcohol Poval JP 18Y, the surfactant Pluronic® F18 from Sigma Aldrich, and the acetylated (I.A.=1.8) and extruded starch also manufactured and sold by the Applicant Company or the xanthan gum sold by the company Sigma Aldrich.

Table II below shows the compositions and the analytical and functional characteristics of the aqueous dispersions thus obtained.

TABLE II
Compositions (%)

| | | | |
|---|---|---|---|
| Galastic ® L68 | 35.5 | 35.4 | 35.0 |
| Triacetin | 8.8 | | |
| Diisobutyl adipate | | 8.8 | |
| Benzoflex 2088 | | | 8.5 |
| Pluronic ® F18 | 0.2 | | |
| Poval JP 18 X polyvinyl alcohol | 1.4 | 1.4 | 1.4 |
| Acetylated extruded starch | 1.4 | 1.0 | 1.0 |
| Xanthan gum | 0.1 | 0.1 | 0.1 |
| Water | 52.6 | 51.3 | 52.0 |
| 10 N alkaline sodium | | 2.0 | 2.0 |
| hydroxide solution | | | |

Initial characteristics

| | | | |
|---|---|---|---|
| Dry matter (%) | 47.2 | 45.0 | 43.0 |
| pH value | 2.2 | 5.0 | 5.0 |
| Mean diameter (μm) | 6.9 | 3.8 | 3.6 |
| d90 (μm) | 19.3 | 9.0 | 8.1 |
| Brookfield viscosity (mPa · s) | 940 | 390 | 950 |
| Appearance | milky | milky | milky |
| | emulsion | emulsion | emulsion |

Characteristics after storage for
2 months at room temperature

| | | | |
|---|---|---|---|
| pH value | 2.1 | 3.6 | 4.2 |
| Brookfield viscosity | 2100 | 760 | 4900 |
| Appearance | milky | milky | creamy |
| | emulsion | emulsion | emulsion |

The aqueous dispersions in accordance with the invention have a low viscosity (strictly less than 1000 mPa.s at 20° C.), a high dry matter content (very much greater than 40%) and excellent stability over time. It should be noted that the aqueous dispersion of PLA prepared with diisobutyl adipate shows noteworthy behaviour over time.

### EXAMPLE 3

An aqueous dispersion in accordance with the invention is prepared according to the process of Example 1, having a composition given in Table III below.

TABLE III
Composition (%)

| | |
|---|---|
| Galastic ® L68 | 46.40 |
| Poval JP 18 Y polyvinyl alcohol | 1.07 |
| Xanthan gum | 0.07 |
| 50/50 mixture of rapeseed methyl | 11.60 |
| ester (Novaol) and of | |
| dipropylene glycol dibenzoate | |
| (Benzoflex ® 9-88) | |
| Water | 40.86 |

Table IV below shows the analytical and functional characteristics of the aqueous dispersion thus obtained, which has a dry matter content of 59.14% by weight.

TABLE IV

| | |
|---|---|
| pH | 2 |
| Brookfield viscosity | |
| (20 rpm, 20° C.) (mPa · s) | |
| initial: | 612 |
| after storage for 1 month: | 1020 |
| Appearance | homogeneous emulsion |
| Coloration | cream-white |
| Laser granulometry (μm) | |
| Mean diameter: | 3.5 |
| d 90 (by volume): | 7.6 |

It was found that this aqueous dispersion free of volatile organic compound in accordance with the invention, when applied as a thin layer to a support, makes it possible to obtain a cohesive film that has excellent water resistance, excellent transparency and high gloss.

1. Aqueous dispersions of at least one biodegradable polymer comprising an organic phase dispersed in a dispersing aqueous phase and free of volatile organic compounds, characterized in that the said organic phase comprises:
at least one viscosity-reducing agent with a solubility parameter of between 15 and 28 (J.cm⁻³)^{0.5}, preferably between 16 and 23 (J.cm⁻³)^{0.5} and even more preferably between 18 and 21 (J.cm⁻³)^{0.5}, and a hydrogen bonding parameter δH of between 3.5 and 15 (J.cm⁻³)^{0.5}, preferably between 4 and 13 (J.cm⁻³)^{0.5} and even more preferably between 5 and 10 (J.cm⁻³)^{0.5},
at least one biodegradable polymer in a polymer/viscosity-reducing agent weight ratio of from 99.8/0.2 to 60/40, preferably between 97/3 and 70/30 and more preferably between 92/8 and 70/30.

2. Dispersions according to claim 1, characterized in that they have a viscosity at 20° C. of between 50 and 5000 mPa.s, preferably between 50 and 2000 mPa.s, even more preferably greater than or equal to 100 mPa.s and strictly less than 1000 mPa.s.

3. Aqueous dispersions of at least one biodegradable polymer comprising an organic phase dispersed in a dispersing aqueous phase and free of volatile organic compounds, characterized in that they have a viscosity at 20° C. of less than 1000 mPa.s., preferably greater than or equal to 100 mPa.s and strictly less than 1000 mPa.s.

4. Dispersions according to any one of claims 1 to 3, characterized in that they have a dry matter content of between 25% and 70% by weight, preferably between 35% and 65% by weight and even more preferably between 40% and 60% by weight.

5. Dispersions according to any one of claims 1 to 4, characterized in that they have a content of biodegradable polymers and of viscosity-reducing agent of at least 80% by weight of the dry matter of the said aqueous dispersions, preferably at least 90% by weight and even more preferably at least 95% by weight.

6. Dispersions according to any one of claims 1 to 5, characterized in that the biodegradable polymer is in amorphous or semi-crystalline form and preferably in amorphous form.

7. Dispersions according to any one of claims 1 to 6, characterized in that the viscosity-reducing agent is chosen from the group of optionally ethoxylated esters of acids and of alcohols, preferably mono-, di- or triesters of organic acids, of carbonic acid and of phosphoric acid, of sugars or of polyols.

8. Dispersions according to any one of claims 1 to 7, characterized in that the viscosity-reducing agent is chosen from the group of optionally ethoxylated ethers of sugars, of polyols, in particular of isosorbide, of glycol or of phenol, of glycol ether-esters or of epoxidized triglyceride oils.

9. Dispersions according to any one of claims 1 to 8, characterized in that the dispersing aqueous phase comprises at least one stabilizer chosen from the group of polyols, oligosaccharides and polysaccharides derived from celluloses, starches and natural and fermenting gums, in native form or modified, preferably by hydroxy-propylation, hydroxy-ethylation, alkylation or alkyl succinylation, peptides and polypeptides, and optionally modified polyvinyl alcohols.

10. Process for preparing aqueous dispersions of at least one biodegradable polymer comprising an organic phase dispersed in a dispersing aqueous phase and free of volatile organic compounds, according to any one of claims 1 to 9, characterized in:
1) preparing the organic phase to be dispersed by melting the blend of biodegradable polymer and of viscosity-reducing agent, at a temperature above the glass transition temperature of the biodegradable polymer so as to obtain a homogeneous melt, the weight ratio between the biodegradable polymer and the viscosity-reducing agent being between 99.8/0.2 and 60/40, preferably between 97/3 and 70/30 and more preferably between 92/8 and 70/30,
2) totally dissolving the stabilizer in the dispersing aqueous phase,
3) simultaneously and continuously introducing the dispersing aqueous phase and the dispersed organic phase thus obtained into the blender of an emulsifying unit, the rates of introduction of the two phases having been adjusted beforehand such that the dispersed organic phase represents at least 80% by weight of the dry matter of the said aqueous dispersions, preferably at least 90% by weight and even more preferably at least 95% by weight, and
4) recovering the aqueous dispersions thus obtained.

11. Use of the aqueous dispersions of at least one biodegradable polymer according to any one of claims 1 to 9 or obtained according to the process of claim 10 for their binding/tacky, printability, degradability, water resistance/behaviour, gloss, oil resistance, stain resistance and mechanical strength properties.

12. Use of the aqueous dispersions of at least one biodegradable polymer according to any one of claims 1 to 9 or obtained according to the process of claim 10 in the fields of textiles, coating fluids, paints, adhesives, renderings and mortar fixatives and coatings/encapsulation.
